(19) United States
(12) Patent Application Publication
(10) Pub. No.: US 2017/0325694 A1
(43) Pub. Date: Nov. 16, 2017

(54) BODY TEMPERATURE MEASURING DEVICE AND METHOD

(71) Applicant: BOE TECHNOLOGY GROUP CO., LTD., Beijing (CN)

(72) Inventor: Hailan JIN, Beijing (CN)

(73) Assignee: BOE TECHNOLOGY GROUP CO., LTD., Beijing (CN)

(21) Appl. No.: 15/514,883

(22) PCT Filed: Sep. 7, 2016
(86) PCT No.: PCT/CN2016/098349

§ 371 Date: Mar. 28, 2017

(30) Foreign Application Priority Data
Jan. 20, 2016 (CN) 201610038930.5

(51) Int. Cl. A61B 5/01; A61B 5/00; G01K 13/00

(52) U.S. Cl. CPC A61B 5/01; A61B 5/6817; A61B 5/0002; G01K 13/00

(57) ABSTRACT

A body temperature measuring device and method. The body temperature measuring device includes: a soft and flexible stick-shaped shell, configured to be inserted into an external auditory canal of a testee in a process of measuring body temperature; a body temperature sensor configured to detect the body temperature; a master control chip connected with the body temperature sensor and configured to determine body temperature data corresponding to the body temperature; and a wireless communication module connected with the master control chip and configured to receive the body temperature data transmitted by the master control chip, and send the body temperature data to an external device. The body temperature sensor, the master control chip and the wireless commutation module are disposed in the stick-shaped shell.

### TECHNICAL FIELD

Embodiments of the present disclosure relate to a body temperature measuring device and method.

### BACKGROUND

As body temperature is closely related to physical conditions, and thus, body temperature measuring devices are widely used. A current type of body temperature measuring device includes mainly thermometers.

However, when a current thermometer is adopted to measure body temperature of a person, it can only detect the temperature once after an interval of time, and the temperature of the human body cannot be continuously acquired in real time. Moreover, when the current thermometer is adopted to measure the body temperature, the thermometer is usually inserted into an armpit, a mouth or an ear of a testee. This not only causes inconvenience to the testee, but also can be easily interfered by external factors, leading to an inaccurate measuring results.

### SUMMARY

Embodiments of the present disclosure provide a body temperature measuring device and method, which can continuously and accurately measure body temperature.

At least one embodiment of the disclosure provides a body temperature measuring device, comprising: a soft and flexible stick-shaped shell, configured to be inserted into an external auditory canal of a testee in a process of measuring body temperature; a body temperature sensor configured to detect the body temperature; a master control chip connected with the body temperature sensor and configured to determine body temperature data corresponding to the body temperature; and a wireless communication module connected with the master control chip and configured to receive the body temperature data transmitted by the master control chip, and send the body temperature data to an external device. The body temperature sensor, the master control chip and the wireless commutation module are disposed in the stick-shaped shell.

For example, in the body temperature measuring device provided in embodiments of the disclosure, the master control chip includes: a signal conversion circuit connected with the body temperature sensor and configured to convert the body temperature detected by the body temperature sensor into the body temperature data; and a first data interface connected with the wireless communication module and configured to transmit the body temperature data to the wireless communication module.

For example, in the body temperature measuring device provided in embodiments of the disclosure, the wireless communication module is also configured to receive a first measuring instruction which includes an acquisition time of the body temperature and is sent by the external device; and the master control chip includes: a second data interface connected with the wireless communication module to acquire the first measuring instruction; and a first control circuit configured to control the acquisition time of the body temperature sensor in acquiring the body temperature according to the first measuring instruction.

For example, in the body temperature measuring device provided in embodiments of the disclosure, the wireless communication module is also configured to receive a second measuring instruction which includes an acquisition frequency of the body temperature and is sent by the external device; and the master control chip includes: a third data interface connected with the wireless communication module to acquire the second measuring instruction; and a second control circuit configured to control the acquisition frequency of the body temperature sensor in acquiring the body temperature according to the second measuring instruction.

For example, in the body temperature measuring device provided in embodiments of the disclosure, the body temperature measuring device further comprises: a power module configured to supply power for the body temperature sensor and the master control chip.

For example, in the body temperature measuring device provided in embodiments of the disclosure, the master control chip includes: a third control circuit configured to provide an alarm instruction when power of the power module is below a threshold; and a fourth data interface connected with the wireless communication module and configured to send the alarm instruction to the wireless communication module, where the wireless communication module is also configured to send the alarm instruction to the external device.

For example, in the body temperature measuring device provided in embodiments of the disclosure, the body temperature measuring device further comprises: a storage chip disposed in the stick-shaped shell, connected with the master control chip, and configured to receive and store the body temperature data transmitted by the master control chip.

For example, in the body temperature measuring device provided in embodiments of the disclosure, the body temperature measuring device further comprises: a hearing aid module disposed in the stick-shaped shell and configured to amplify external sound.

For example, in the body temperature measuring device provided in embodiments of the disclosure, the body temperature sensor is an infrared body temperature sensor.

For example, in the body temperature measuring device provided in embodiments of the disclosure, the stick-shaped shell is made from polyethylene, polypropylene, polyvinyl chloride or silica gel.

For example, in the body temperature measuring device provided in embodiments of the disclosure, a length of the stick-shaped shell is between 10 mm and 20 mm, and a sectional dimension of the stick-shaped shell is between 5 mm*5 mm and 10 mm*10 mm.

Embodiments of the disclosure also provide a body temperature measuring method employing the body temperature measuring device described above. The method includes: detecting body temperature by a body temperature sensor; acquiring, by a master control chip, body temperature data corresponding to the body temperature, and sending the body temperature data to a wireless communication module; and sending the received body temperature data to an external device by the wireless communication module.

### BRIEF DESCRIPTION OF THE DRAWINGS

FIG. 1 is a schematic diagram illustrating application of a body temperature measuring device provided by an embodiment of the present disclosure; and

FIG. 2 is a schematic view of a structure of a body temperature measuring device provided by an embodiment of the present disclosure.

Reference numerals of the accompanying drawings:

1. infrared body temperature sensor 2. power module 3. master control chip 4. storage chip 5. hearing aid module 6. wireless communication module

### DETAILED DESCRIPTION

Hereafter, the technical solutions of the embodiments of the present disclosure will be described in a clearly and fully understandable way in connection with the drawings related to the embodiments of the disclosure. It is obvious that the described embodiments are just a part but not all of the embodiments of the present disclosure. Based on embodiments of the present disclosure, all other embodiments obtained by those skilled in the art without making other inventive work should be within the scope of the present disclosure.

Unless otherwise clearly defined, technological terms or scientific terms used in the disclosure have common meanings understood by persons of ordinary skills in a field that the disclosure belongs to. In the present disclosure, terms such as “first”, “second” and the like used in the present disclosure do not indicate any sequence, quantity or significance but only for distinguishing different constituent parts. The terms “comprises,” “comprising,” “includes,” “including,” etc., are intended to specify that the elements or the objects stated before these terms encompass the elements or the objects and equivalents thereof listed after these terms, but do not preclude the other elements or objects. Terms such as “coupled”, “connected” should be broadly interpreted, for example, it may be fixedly connected, or may be detachably connected, or integrally connected; it may be mechanically connected, or may be electrically connected; it may be directly connected, or may be indirectly connected via an intermediate medium. Terms such as “up,” “down,” “left,” “right” and so on, are used to indicate relative positional relationships. When an absolute position of a described object changes, a relative positional relationship of the object also changes possibly.

### First Embodiment

The embodiment provides a body temperature measuring device. The body temperature measuring device comprises: a soft and flexible stick-shaped shell, configured to be inserted into an external auditory canal of a testee in a process of measuring body temperature; a body temperature sensor configured to detect the body temperature; a master control chip connected with the body temperature sensor and configured to determine body temperature data corresponding to the body temperature; and a wireless communication module connected with the master control chip and configured to receive the body temperature data transmitted by the master control chip and send the body temperature data to an external device. The body temperature sensor, the master control chip and the wireless communication module are disposed inside the stick-shaped shell.

In the embodiment, the shell of the body temperature measuring device is soft and flexible. In the process of measuring the body temperature, the entire body temperature measuring device is disposed inside an external auditory canal of a testee and can measure the temperature of the eardrum. Compared with a shell temperature, the eardrum temperature is closer to a core temperature. Moreover, the entire body temperature measuring device is disposed inside the external auditory canal of the testee, may not move or change angles, is not susceptible to external interference, and can continuously and accurately measure the body temperature. The body temperature measuring device communicates with the external device through the wireless communication module, may receive an external instruction and measure the body temperature according to the external instruction, and may also send the body temperature data to the external device, so as to achieve storage and further processing of the body temperature data.

For instance, the master control chip includes: a signal conversion circuit connected with the body temperature sensor and configured to convert the body temperature detected by the body temperature sensor into the body temperature data; and a first data interface connected with the wireless communication module and configured to transmit the body temperature data to the wireless communication module.

For instance, the wireless communication module is also configured to receive a first measuring instruction which includes an acquisition time of the body temperature and is sent by the external device. The master control chip includes a second data interface connected with the wireless communication module, so as to acquire the first measuring instruction; and a first control circuit, configured to control a measuring time of the body temperature sensor in acquiring the body temperature according to the first measuring instruction.

For instance, the wireless communication module is also configured to receive a second measuring instruction which includes an acquisition frequency of the body temperature and is sent by the external device. The master control chip includes: a third data interface connected with the wireless communication module, so as to acquire the second measuring instruction; and a second control circuit configured to control a measuring frequency of the body temperature sensor in acquiring the body temperature according to the second measuring instruction.

For instance, the body temperature measuring device further comprises: a hearing aid module disposed in the stick-shaped shell and configured to amplify external sound. Thus, when the body temperature measuring device is placed in the external auditory canal of the testee, receipt of the external sound may not be affected for the testee.

For instance, the body temperature sensor is an infrared body temperature sensor.

For instance, the soft and flexible stick-shaped shell may be made from polyethylene, polypropylene, polyvinyl chloride or silica gel.

For instance, in order to place the body temperature measuring device into the external auditory canal of the testee, a length of the stick-shaped shell is 10 mm-20 mm, and a sectional dimension of the stick-shaped shell is 5 mm*5 mm-10 mm*10 mm.

### Second Embodiment

The embodiment provides a body temperature measuring method employing the body temperature measuring device described above. The method comprises: detecting body temperature by the body temperature sensor; acquiring, by the master control chip, body temperature data corresponding to the body temperature, and sending the body temperature data to the wireless communication module; and sending, by the wireless communication module, the received body temperature data to the external device.

In the embodiment, the shell of the body temperature measuring device is soft and flexible. In the process of measuring the body temperature, the entire body temperature measuring device is disposed in the external auditory canal of the testee and can measure the temperature of the eardrum. Compared with the shell temperature, the eardrum temperature is closer to the core temperature. Moreover, the entire body temperature measuring device is disposed in the external auditory canal of the testee, may not move or change the angles, is not susceptible to external interference, and can continuously and accurately measure the body temperature. The body temperature measuring device communicates with the external device through the wireless communication module, may receive an external instruction and measure the body temperature according to the external instruction, and may also send the body temperature data to the external device, so as to achieve storage and further processing of the body temperature data.

### Third Embodiment

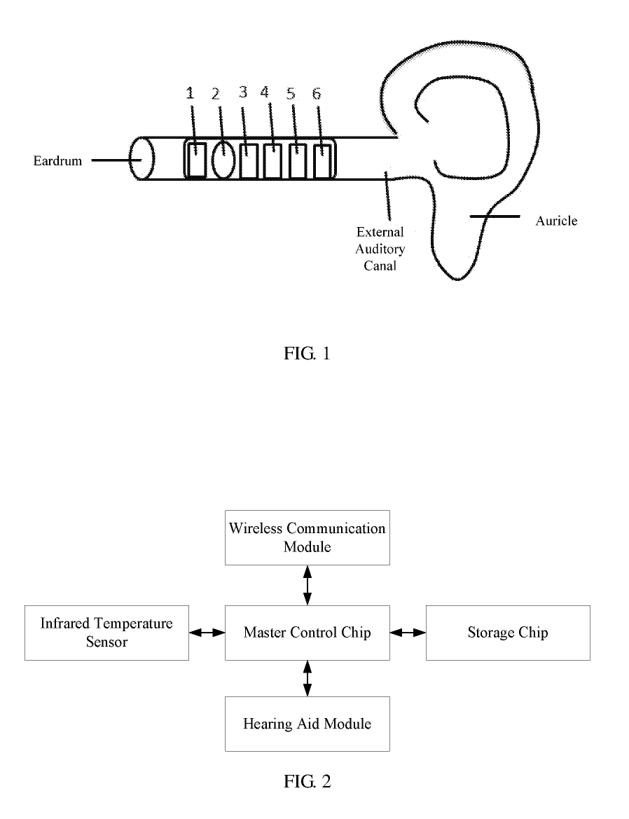

The embodiment provides a body temperature measuring device. As illustrated in FIGS. 1 and 2, the body temperature measuring device comprises a soft and flexible stick-shaped shell, and an infrared body temperature sensor 1, a power module 2, a master control chip 3, a storage chip 4, a hearing aid module 5 and a wireless communication module 6 which are disposed in the stick-shaped shell, where: the master control chip 3 is connected with the infrared body temperature sensor 1, the wireless communication module 6, the storage chip 4 and the hearing aid module 5; and the power module 2 is configured to supply power for the infrared body temperature sensor 1, the master control chip 3, the hearing aid module 5 and the wireless communication module 6.

For instance, the infrared body temperature sensor 1 is packaged by a 1.6 mm×1.6 mm wafer chip, e.g., a tmp006 chip; the master control chip 3 may be packaged using thin quad flat package (TQFP), with a thickness not greater than 1 mm, a pitch being 0.5 mm, and both a length and a width being controlled to be within 3 mm; the power module 2 adopts flexible cells, for instance, may be manufactured by a liquid-metal printing technology; an antenna of the wireless communication module 6 may also be manufactured by a liquid-metal printing technology; the hearing aid module 5 includes a sound transmission circuit and a hearing aid circuit, in which the sound transmission circuit is disposed at one end of a porus acusticus externus in the auditory canal, and the hearing aid circuit is disposed at one end of the eardrum in the auditory canal; and the stick-shaped shell may be made from flexible materials such as polyethylene, polypropylene, polyvinyl chloride and silica gel.

For instance, the body temperature measuring device provided by the embodiment may be manufactured by 3D printing technologies. The printing of the shell and the printing of the circuits are simultaneously performed; and the material of the circuits may adopt liquid metals or other conductive materials. The placing of the master control chip, the storage chip and the like may be manually performed or may be executed by a mechanical arm so as to achieve more accurate control effect. When a liquid metal is adopted as the material of the circuits, connection between the liquid metal and chip pins does not require welding, so that the softness and the flexibility of the body temperature measuring device can be further enhanced. Moreover, a waterproof layer may also be disposed on the outside of the body temperature measuring device, so that the body temperature may also be measured when the testee takes a shower or exercises under water.

In the embodiment, a volume of the body temperature measuring device is controlled to be between 5 mm*5 mm*10 mm and 10 mm*10 mm*20 mm. Thus, in the process of measuring the body temperature, the stick-shaped shell can be inserted into the external auditory canal of the testee. The testee may select to wear the body temperature measuring device provided by the embodiment in one ear or two ears.

In the process of measuring the body temperature, the infrared body temperature sensor 1 detects the body temperature at the eardrum of the testee; the master control chip 3 converts the body temperature detected by the infrared body temperature sensor 1 into the body temperature data, and transmits the body temperature data to the wireless communication module 6 and the storage chip 4; the storage chip 4 may store the obtained body temperature data; the wireless communication module 6 may send the body temperature data to the external device, e.g., a mobile phone or a computer; and moreover, the wireless communication module 6 may also send the body temperature data to a server, and a personal body temperature database can be created in the server.

For instance, the external device may also set the acquisition time and the acquisition frequency of the body temperature measuring device in acquiring the body temperature through the wireless communication module 6. For instance, the wireless communication module 6 receives a first measuring instruction which includes an acquisition time of the body temperature and is sent by the external device, and the master control chip 3 controls the acquisition time of the infrared body temperature sensor 1 in acquiring the body temperature according to the first measuring instruction; or the wireless communication module 6 receives a second measuring instruction which includes an acquisition frequency of the body temperature and is sent by the external device, and the master control chip 3 controls the acquisition frequency of the infrared body temperature sensor 1 in acquiring the body temperature according to the second measuring instruction.

In the embodiment, the shell of the body temperature measuring device is soft and flexible. In the process of measuring the body temperature, the entire body temperature measuring device is disposed in the external auditory canal of the testee and can measure the temperature of the eardrum. Compared with the shell temperature, the eardrum temperature is closer to the core temperature. Moreover, the entire body temperature measuring device is disposed in the external auditory canal of the testee, may not move or change the angle, is not susceptible to external interference, and can continuously and accurately measure the body temperature. The body temperature measuring device communicates with the external device through the wireless communication module, may receive an external instruction and measure the body temperature according to the external instruction, and may also send the body temperature data to the external device, so as to achieve the storage and further processing of the body temperature data.

In a case such as fever of an infant or toddler, the body temperature measuring device provided by the embodiment may be adopted to solve problems such as noncooperation of the infant or toddler in the body temperature measurement and inconvenience in multiple measurements. As for women preparing for pregnancy and women in pregnancy, the body temperature measuring device provided by the embodiment may be adopted to continuously and accurately measure the body temperature, so that a basal body temperature curve can be drawn, and hence the ovulation days can be accurately predicted or the health in pregnancy can be monitored. As for the elderly, the body temperature measuring device provided by the embodiment may be adopted to send body temperature data to external devices of their children, so that the children can know the health condition of the elderly.

Many functional units illustrated in the description are all referred to as modules, so as to emphasize independence of their implementations.

In the embodiments of the present disclosure, the modules may be implemented by software, so as to be executed by various types of processors. For instance, an identified executable code module may include one or more physical or logical blocks of computer instructions, and may be created as an object, a process or a function, etc. In spite of this, executable codes of the identified modules are not required to be physically set together but may include different instructions stored on different physical blocks. When the instructions are logically combined, a module is created and a specific objective of the module can be achieved.

Actually, an executable code module may include a single instruction or many instructions, and even may be distributed on a plurality of different code segments, distributed in different programs, and distributed in a plurality of storage devices. Similarly, operating data may be identified in modules, may be implemented by any appropriate form and constructed in any suitable type of data structure. The operating data may be collected as a single data set, or may be distributed at different positions (on different storage devices), and at least one part of the operating data may be taken as electric signals and stored on a system or network.

When the modules can be implemented by software, in consideration of the level of the conventional hardware technology, as for the modules capable of being implemented by software, corresponding hardware circuits may be created by those skilled in the art to achieve corresponding functions without regard to cost. The hardware circuits include conventional very large scale integration (VLSI) circuits or gate arrays and semiconductors such as logic chips and transistors or other discrete components. The modules may also be implemented by programmable hardware units such as field programmable gate array (FPGA), programmable logic array and programmable logic device.

The body temperature device and method, provided by the embodiments of the present disclosure, have the following advantages: the shell of the body temperature measuring device is soft and flexible, so that in the process of measuring the body temperature, the entire body temperature measuring device is disposed in the external auditory canal of the testee and can measure the temperature of the eardrum; compared with the shell temperature, the eardrum temperature is closer to the core temperature; moreover, the entire body temperature measuring device is disposed in the external auditory canal of the testee, may not move or change the angle, is not susceptible to external interference, and can continuously and accurately measure the body temperature; the body temperature measuring device communicates with the external device through the wireless communication module, may receive an external instruction and measure the body temperature according to the external instruction, and may also send the body temperature data to the external device, so as to achieve the storage and further processing of the body temperature data.

In the method embodiments of the present disclosure, the serial numbers of the steps are not intended to define the sequence of the steps. The change in the sequence of the steps made by those skilled in the art without creative efforts shall also fall within the scope of protection of the present disclosure.

What are described above is related to the illustrative embodiments of the disclosure only and not limitative to the scope of the disclosure. The scopes of the disclosure are defined by the accompanying claims.

The present application claims the priority of the Chinese Patent Application No. 201610038930.5 filed on Jan. 20, 2016, which is incorporated herein by reference in its entirety as part of the disclosure of the present application.

1. A body temperature measuring device, comprising:
a soft and flexible stick-shaped shell, configured to be inserted into an external auditory canal of a testee in a process of measuring body temperature;
a body temperature sensor configured to detect the body temperature;
a master control chip connected with the body temperature sensor and configured to determine body temperature data corresponding to the body temperature; and
a wireless communication module connected with the master control chip and configured to receive the body temperature data transmitted by the master control chip, and send the body temperature data to an external device,
wherein the body temperature sensor, the master control chip and the wireless commutation module are disposed in the stick-shaped shell.

2. The body temperature measuring device according to claim 1, wherein the master control chip includes:
a signal conversion circuit connected with the body temperature sensor and configured to convert the body temperature detected by the body temperature sensor into the body temperature data; and
a first data interface connected with the wireless communication module and configured to transmit the body temperature data to the wireless communication module.

3. The body temperature measuring device according to claim 1, wherein the wireless communication module is also configured to receive a first measuring instruction which includes an acquisition time of the body temperature and is sent by the external device; and
the master control chip includes:
a second data interface connected with the wireless communication module to acquire the first measuring instruction; and
a first control circuit configured to control the acquisition time of the body temperature sensor in acquiring the body temperature according to the first measuring instruction.

4. The body temperature measuring device according to claim 1, wherein:
the wireless communication module is also configured to receive a second measuring instruction which includes an acquisition frequency of the body temperature and is sent by the external device; and
the master control chip includes:
a third data interface connected with the wireless communication module to acquire the second measuring instruction; and
a second control circuit configured to control the acquisition frequency of the body temperature sensor in acquiring the body temperature according to the second measuring instruction.

5. The body temperature measuring device according to claim 1, wherein the body temperature measuring device further comprises: a power module configured to supply power for the body temperature sensor and the master control chip.

6. The body temperature measuring device according to claim 5, wherein the master control chip includes:
a third control circuit configured to provide an alarm instruction when power of the power module is below a threshold; and
a fourth data interface connected with the wireless communication module and configured to send the alarm instruction to the wireless communication module,
wherein the wireless communication module is also configured to send the alarm instruction to the external device.

7. The body temperature measuring device according to claim 1, wherein the body temperature measuring device further comprises:
a storage chip disposed in the stick-shaped shell, connected with the master control chip, and configured to receive and store the body temperature data transmitted by the master control chip.

8. The body temperature measuring device according to claim 1, wherein the body temperature measuring device further comprises: a hearing aid module disposed in the stick-shaped shell and configured to amplify external sound.

9. The body temperature measuring device according to claim 1, wherein the body temperature sensor is an infrared body temperature sensor.

10. The body temperature measuring device according to claim 1, wherein the stick-shaped shell is made from polyethylene, polypropylene, polyvinyl chloride or silica gel.

11. The body temperature measuring device according to claim 1, wherein a length of the stick-shaped shell is between 10 mm and 20 mm, and a sectional dimension of the stick-shaped shell is between 5 mm*5 mm and 10 mm*10 mm.

12. A body temperature measuring method employing the body temperature measuring device according to claim 1, comprising:
detecting body temperature by a body temperature sensor;
acquiring, by a master control chip, body temperature data corresponding to the body temperature, and sending the body temperature data to a wireless communication module; and
sending the received body temperature data to an external device by the wireless communication module.

13. The body temperature measuring device according to claim 2, wherein the body temperature measuring device further comprises:
a storage chip disposed in the stick-shaped shell, connected with the master control chip, and configured to receive and store the body temperature data transmitted by the master control chip.

14. The body temperature measuring device according to claim 2, wherein the body temperature measuring device further comprises: a hearing aid module disposed in the stick-shaped shell and configured to amplify external sound.

15. The body temperature measuring device according to claim 2, wherein the body temperature sensor is an infrared body temperature sensor.

16. The body temperature measuring device according to claim 2, wherein the stick-shaped shell is made from polyethylene, polypropylene, polyvinyl chloride or silica gel.

17. The body temperature measuring device according to claim 2, wherein a length of the stick-shaped shell is between 10 mm and 20 mm, and a sectional dimension of the stick-shaped shell is between 5 mm*5 mm and 10 mm*10 mm.

18. The body temperature measuring device according to claim 3, wherein the body temperature measuring device further comprises:
a storage chip disposed in the stick-shaped shell, connected with the master control chip, and configured to receive and store the body temperature data transmitted by the master control chip.

19. The body temperature measuring device according to claim 3, wherein the body temperature measuring device further comprises: a hearing aid module disposed in the stick-shaped shell and configured to amplify external sound.

20. The body temperature measuring device according to claim 3, wherein the body temperature sensor is an infrared body temperature sensor.
